(19) United States
(12) Patent Application Publication
(10) Pub. No.: US 2008/0154131 A1
(43) Pub. Date: Jun. 26, 2008

(54) METHODS FOR ENHANCEMENT OF VISIBILITY OF ABLATION REGIONS

(75) Inventors: WARREN LEE, NISKAYUNA, NY (US); MIRSAID SEYED-BOLORFOROSH, GUILDERLAND, NY (US); AARON MARK DENTINGER, LATHAM, NY (US); KAI ERIK THOMENIUS, CLIFTON PARK, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: GENERAL ELECTRIC COMPANY, SCHENECTADY, NY (US)

(21) Appl. No.: 11/613,217

(22) Filed: Dec. 20, 2006

(51) Int. Cl. A61B 8/00

(52) U.S. Cl. 600/439

(57) ABSTRACT

A method for imaging during ablation procedures using ultrasound imaging is provided. The method includes obtaining input image data about an ablation region, wherein the image data comprises back scatter intensity, and applying a dynamic gain curve based on the image data to obtain an output signal for use in enhancing the visibility of the ablation region.

### BACKGROUND

The invention relates generally to diagnostic imaging, and more particularly to enhancement of visibility in ablation regions.

Heart rhythm problems or cardiac arrhythmias are a major cause of mortality and morbidity. Atrial fibrillation is one of the most common sustained cardiac arrhythmias encountered in clinical practice. Cardiac electrophysiology has evolved into a clinical tool to diagnose and treat these cardiac arrhythmias. As will be appreciated, during electrophysiological studies, multipolar catheters are positioned inside the anatomy, such as the heart, and electrical recordings are made from different locations inside the heart. Further, catheter-based ablation therapies have been employed for the treatment of atrial fibrillation.

Conventional techniques utilize radio frequency (RF) catheter ablation for the treatment of atrial fibrillation. Currently, catheter placement within the anatomy is typically performed under fluoroscopic guidance. Intracardiac echocardiography has also been employed during RF catheter ablation procedures. Additionally, the ablation procedure may necessitate the use of a multitude of devices, such as a catheter to form an electroanatomical map of the anatomy, such as the heart, a catheter to deliver the RF ablation, a catheter to monitor the electrical activity of the heart, and an imaging catheter. A drawback of these techniques however is that these procedures are extremely tedious requiring considerable manpower, time and expense. Further, the long procedure times associated with the currently available catheter-based ablation techniques increase the risks associated with long term exposure to ionizing radiation to the patient as well as medical personnel.

There are several treatments available for individuals with abnormal cardiac electrical activity such as atrial fibrillation. One increasingly popular invasive treatment is catheter ablation. During such procedures, catheters are guided into the heart and energy in the form of radiofrequency, cryo, laser or other types, are delivered to the tissue(s) responsible for the arrhythmia. Localized destruction of the tissue supporting the abnormal cardiac electrical activity results, thus restoring normal sinus rhythm.

Currently, many of these ablation procedures utilize an electroanatomical mapping system, in which a mapping catheter is used to acquire a static map of the desired region prior to ablation, and the ablation locations are recorded onto the static map as they are generated. Unfortunately, acquisition of the static map is very time consuming, and both the depicted anatomy and ablation locations are often inaccurate due to the dynamic nature of the beating heart. Typically, there is an increase in the echogenicity of ablated regions compared to non-ablated regions. However, these differences are often subtle and difficult to detect using conventional ultrasound imaging systems. Methods that are capable of identifying the size and location of the ablation lesions on an actual dynamic image of the heart would increase both the accuracy as well as the efficiency of ablation procedures.

There is therefore a need for systems and methods that allow ablation regions to be more readily visualized, thus allowing the ablation procedure to be monitored in real-time on a dynamic image, thereby increasing the accuracy and efficiency of ablation procedures.

### BRIEF DESCRIPTION

In one embodiment of the present technique, a method for imaging during ablation procedures using ultrasound imaging is provided. The method includes obtaining input image data about an ablation region, wherein the image data comprises back scatter intensity, and applying a dynamic gain curve based on the image data to obtain an output signal for use in enhancing the visibility of the ablation region.

In another embodiment of the present technique, a method for enhancing the visibility of an ablation region during ablation procedures is provided. The method includes processing backscatter data from one or more image frames to identify changes in localized regions of image data, and applying a dynamic gain curve to obtain an enhanced output signal from the ablation region.

In yet another embodiment of the present technique, a method for in-situ enhancement of the visibility of an ablation region is provided. The method includes monitoring the ablation region, tracking a location of a catheter tip during ablation in the ablation region, analyzing a backscatter intensity in a predetermined region around the catheter tip, and adjusting the system settings to obtain enhanced backscatter data from the predetermined region.

### DRAWINGS

These and other features, aspects, and advantages of the present invention will become better understood when the following detailed description is read with reference to the accompanying drawings in which like characters represent like parts throughout the drawings, wherein:

FIG. 1 is a block diagram of an exemplary ultrasound imaging system, in accordance with aspects of the present technique;

FIG. 2 is a block diagram illustrating an exemplary method for enhancement of visibility of the ablation region, in accordance with aspects of the present technique;

FIG. 3 is a block diagram illustrating the functional steps employed by the processor of FIG. 2, in accordance with aspects of the present technique;

FIGS. 4-6 are graphical representations of exemplary dynamic gain curves being applied to the image data, in accordance with aspects of the present technique;

FIGS. 7-9 are schematics illustrating the change in visibility in an ablation region on applying the different dynamic gain curves, in accordance with aspects of the present technique;

FIG. 10 is a block diagram illustrating an exemplary method for applying a dynamic gain curve to the image data acquired from an ablation region, in accordance with aspects of the present technique;

FIG. 11 is a block diagram illustrating an exemplary method for enhancing the visibility of an ablation region by tracking a trip of the catheter, in accordance with aspects of the present technique;

FIGS. 12-13 illustrate an ablation region before and after applying the dynamic gain curve, respectively, in accordance with aspects of the present technique;

FIG. 14 is a block diagram illustrating an exemplary method for enhancing the visibility of the ablation region by recording pre and post-ablation regions, in accordance with aspects of the present technique;

FIG. 15 illustrates the pre- and post ablation image frames, in accordance with aspects of the present technique;

FIG. 16 illustrates a two step process for calculation of the gain curve, in accordance with aspects of the present technique; and

FIG. 17 illustrates enhancement of the visibility of the ablation region upon application of the gain curve using the two step process of FIG. 16.

### DETAILED DESCRIPTION

As will be described in detail hereinafter, ultrasound imaging systems and methods for real-time monitoring of ablation procedures and ablated regions in accordance with exemplary aspects of the present technique are presented. The systems and methods are configured to enhance visibility of the ablation regions in ultrasound imaging. As used herein, the term “ablation region” refers to a target volume affected by one or more of RF ablation, cryogenic ablation, chemical ablation, focused ultrasound beam, for example, employed to affect tissues in the target volume. Real-time, dynamic ablation monitoring systems represent a significant advancement beyond the static monitoring systems such as the CARTO electroanatomical mapping currently in use. The systems and methods described hereinafter may be employed in different types of ultrasound probes including intercardiac, transesophageal, transthoracic probes, and is applicable to all different types of ablation procedures using both internal (e.g., catheter) and external (e.g., High Intensity Focused Ultrasound (HIFU) ablation devices. It should be appreciated that HIFU devices may also be internal. Also, the present technique may be applied to different locations, such as heart, liver. Further, the present technique may be employed for either two dimensional (2D) or three dimensional (3D) images. The image data may be acquired in real-time employing the imaging catheter. This acquisition of image data via the imaging catheter aids a user in guiding the imaging catheter or ablation device to a desirable location. It should be noted that mechanical means, electronic means, or both may be employed to facilitate the acquisition of image data via the imaging catheter. The imaging catheter may include an imaging transducer. Alternatively, previously stored image data representative of the anatomical region may be acquired by the imaging system. Further, the ablation may be facilitated by employing one or more of ethanol, liquid nitrogen, ultrasound or radio frequency radiation. In an exemplary embodiment, ethanol may be employed for chemical ablation of the tissues, the liquid nitrogen may be employed to cryogenically freeze the ablation tissue, and the ultrasound or radio frequency radiation may be employed to burn the tissues.

Although, the exemplary embodiments illustrated hereinafter are described in the context of a medical imaging system, it will be appreciated that use of the ultrasound imaging system in industrial applications are also contemplated in conjunction with the present technique.

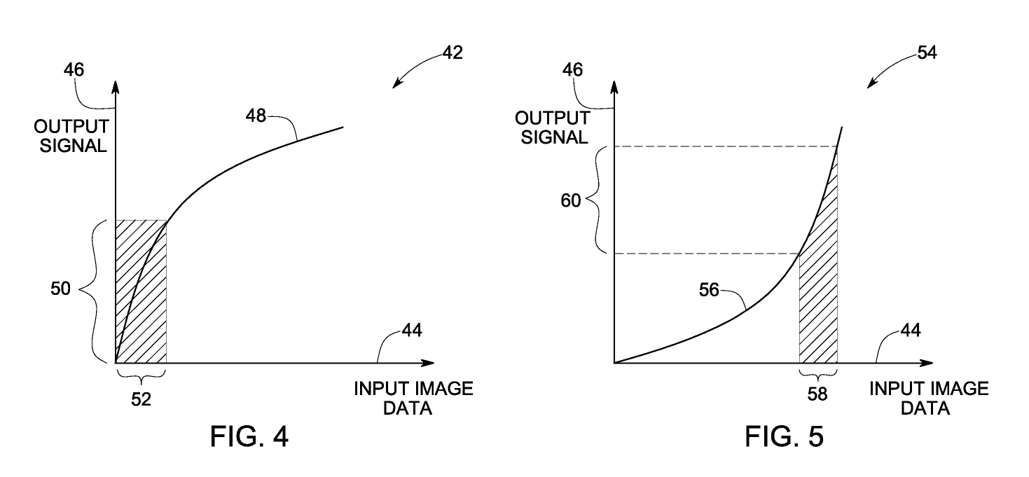

In certain embodiments, a method for imaging during ablation includes obtaining input image data about an ablation region. The image data embodies a range of data or a single value. For example, the image data may include backscatter properties. As used herein, the term “backscatter properties” is broadly used to refer to radiation/signals emitted by the ablated tissues during ablation. The visibility of the ablation region is enhanced by applying one or more dynamic curves based on the input image data to obtain enhanced output signal, as will be described in detail below with regard to FIGS. 4-9. The term “dynamic gain curve” encompasses any curves or equations that may be applied to the input image data to generate output signals that can be displayed by the imaging system. Also, the term “dynamic” in the dynamic gain curve represents the dynamic nature of the curve during the evaluation of the visibility of an ablation region. In other words, the gain curve may be altered if the visibility of the ablation region is not enhanced to a desirable level. Further, system settings may be applied to enhance the visibility of the ablation region. Further, the system settings may either be applied to the entire displayed image, or the system settings may take effect only in the region of interest, which forms a portion of the entire displayed image. As used herein, the term “system settings” or “system display settings” is broadly used to refer to any parameters of the ultrasound imaging system that affect the display of acquired image data.

As will be described in detail below, the ablation region may be identified in different ways. In certain embodiments, the region from where the image data is obtained is selected by tracking the tip of the catheter. In these embodiments, the backscatter intensity is obtained from a predetermined region around the catheter tip. In other embodiments, the image data is calculated by comparing pre- and post-ablation images. Also, the image data may be obtained either from the entire ablation region or from a selected portion of the ablation region.

In certain embodiments, the ultrasound imaging system processes image data from one or more image frames containing a region with ablated tissues, and based upon altered backscatter properties of the ablated tissue, automatically selects system settings to improve the visibility of the ablated tissues, thereby allowing a user to more accurately and efficiently conduct ablation procedures. In some embodiments, the image data from the one or more image frames may be integrated to account for the spatial movement of the ablated tissues.

In some embodiments, an ultrasound imaging system tracks the location of a tip of the one or more ablation catheters. Subsequently, image data in a predetermined region around the tip locations having ablated issues is analyzed. Subsequently, a dynamic gain curve is applied to the image data in the predetermined region. Further, the system settings may be selected so as to improve the visibility of the ablated tissues in a selected region around the catheter tip.

In other embodiments, an ultrasound imaging system acquires and stores image frames prior to an ablation as well as after the ablation, registers the image frames, and analyzes differences in the registered images in order to display data corresponding to echogenicity changes due to ablated tissues.

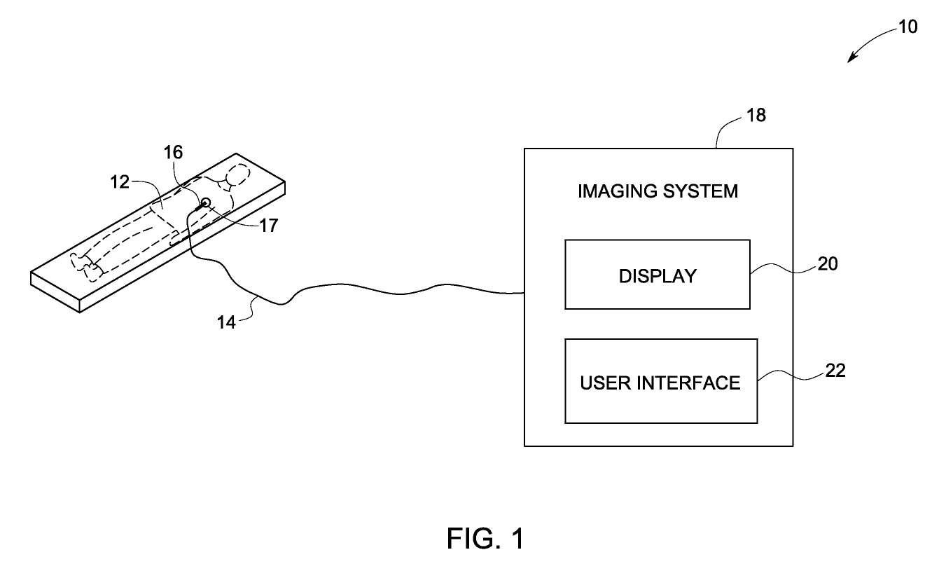

FIG. 1 is a block diagram of an exemplary system 10 for use in guiding a probe in accordance with aspects of the present technique. It should be noted that the figures are for illustrative purposes and are not necessarily drawn to scale. The system 10 may be configured to facilitate acquisition of image data from a patient 12 via a probe 14. In other words, the probe 14 may be configured to acquire image data representative of a region of interest in the patient 12, for example. In accordance with aspects of the present technique, the probe 14 may be configured to facilitate interventional procedures. It should also be noted that although the embodiments illustrated are described in the context of a catheter-based probe, other types of probes such as endoscopes, laparoscopes, surgical probes, probes adapted for interventional procedures, or combinations thereof are also contemplated in conjunction with the present technique. Reference numeral 16 is representative of a portion of the probe 14 disposed inside the vasculature of the patient 12.

In certain embodiments, the probe may include an imaging catheter-based probe 14. Further, an imaging orientation of the imaging catheter 14 may include a forward viewing catheter or a side viewing catheter. However, a combination of forward viewing and side viewing catheters may also be employed as the imaging catheter 14. The imaging catheter 14 may include a real-time imaging transducer (not shown).

As previously noted, the imaging catheter 14 may be configured to facilitate ablation of a region and for acquisition of image data from the patient 12. As described in detail below, in accordance with aspects of the present technique, the imaging catheter 14 may be configured to facilitate tracking of the ablation region 17 within the vasculature of the patient 12.

The system 10 may also include an imaging system 18 that is in operative association with the imaging catheter 14 and configured to facilitate tracking of the ablation region 17. In one embodiment, the imaging system 18 is configured to actively guide the catheter 14 to the ablation region 17 or physically locate the tip of the catheter 14. In another embodiment, a clinician may manually guide the catheter 14 based on the images. In this embodiment, the tracking of the ablation region 17 is achieved by monitoring specific features of the images, such as the catheter tip, or the tissue. Once the location of the ablation catheter tip is recognized, the visibility of the ablation region 17 may be enhanced by applying specific system settings, such as the gain curve, to a region around the tip.

In accordance with aspects of the present technique, the imaging system 18 may be configured to generate a current image based on the acquired image data. As used herein, “current” image embodies an image representative of the current position of the imaging catheter 14. Accordingly the imaging system 18 may be configured to acquire image data representative of an anatomical region of the patient 12 via the imaging catheter 14. While image data may be directly acquired from the patient 12 via the imaging catheter 14, the imaging system 18 may instead acquire stored image data representative of the anatomical region of the patient 12 from an archive site or data storage facility.

Further, the imaging system 18 may be configured to display the generated image representative of a current position of the imaging catheter 14 within a region of interest in the patient 12. As illustrated in FIG. 1, the imaging system 18 may include a display area 20 and a user interface area 22. In accordance with aspects of the present technique, the display area 20 of the imaging system 18 may be configured to display the image generated by the imaging system 18 based on the image data acquired via the imaging catheter 14. Additionally, the display area 20 may be configured to aid the user in visualizing the generated image.

Further, the user interface area 22 of the imaging system 18 may include a human interface device (not shown) configured to facilitate the user to manipulate the guidance of the imaging catheter 14 within the vasculature of the patient 12. The human interface device may include a mouse-type device, a trackball, a joystick, or a stylus. However, as will be appreciated, other human interface devices, such as, but not limited to, a touch screen, may also be employed.

Additionally, a larger context to aid in the visualization of the ablation region 17 and guidance of the imaging catheter 14 to the second ablation region, once the therapy has been delivered at the first ablation region, may be provided by coalescing the images generated based on image data acquired via the imaging catheter 14 with previously acquired images of the anatomical region being imaged. Accordingly, the imaging system 18 may also include a workstation (not shown) configured to register the generated images with previously acquired images of the region of interest being imaged. The previously acquired images may include images acquired via a variety of imaging techniques including, but not limited to, a computed tomography (CT) image, a magnetic resonance image (MR), an X-ray image, a nuclear medicine image, a positron emission tomography (PET) image, images acquired via other developing techniques, or combinations thereof. Additionally, the workstation may be configured to display the registered images on the display area 20 of the imaging system 18.

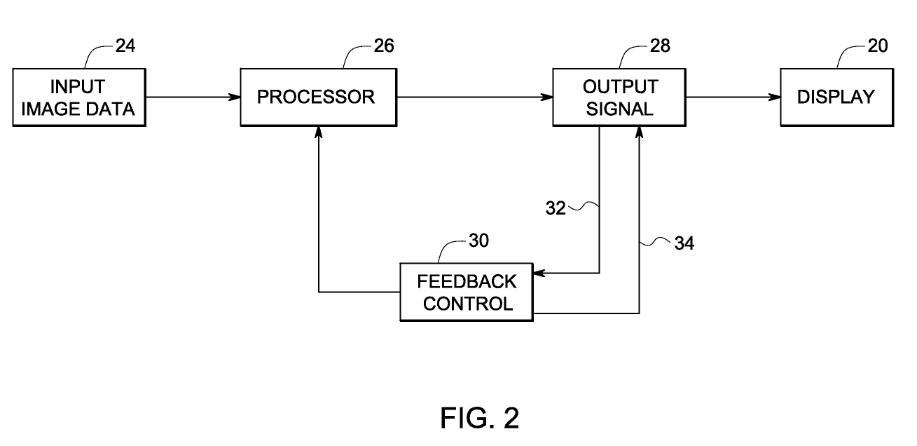

FIG. 2 is an illustration of a method of enhancing visibility of an ablation region, in accordance with aspects of the present technique. Input image data is obtained about an ablation region, the image data includes backscatter intensity from the ablation region.

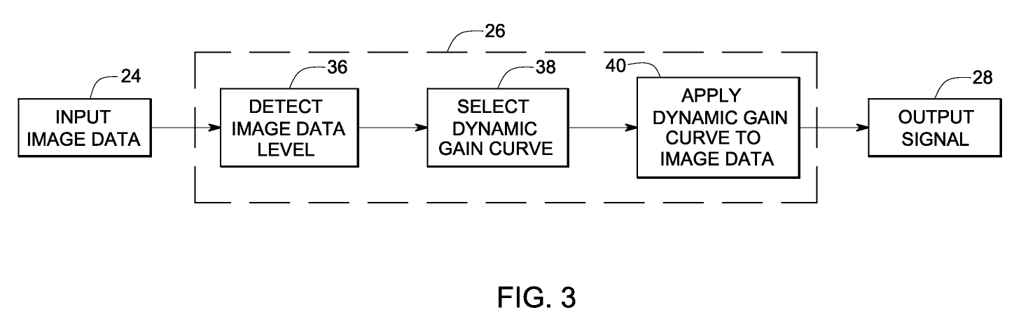

As depicted in FIG. 2, the input image data 24 is obtained from an ablation region. The input image data 24 serves as an input for the processor 26. In response to the input image data 24, the processor 26 employs a dynamic gain curve to produce an output signal 28, such that the output signal 28 enhances the visibility in the ablation region. Additionally, the processor 26 may also alter the system settings to further enhance the visibility of the ablation region. In an exemplary embodiment, the visibility in the ablation region may be enhanced by applying a gain curve which increases the contrast between the ablated and the non-ablated tissues as will be described in detail with regard to FIGS. 3-6. As described in detail below with regard to FIG. 3, the processor 26 selects or employs a dynamic gain curve based on the corresponding value of the image data.

As illustrated, the output signal 28 generated by the application of the dynamic gain curve may then be displayed at display 20. In certain embodiments, once the dynamic gain curve is selected, the output signal 28 may be evaluated, if the output signal 28 is found to be sufficient to enhance the visibility of the ablation region to a desirable level, the dynamic gain curve is retained, else, a different dynamic gain curve may be applied for the same input image data 24. The functioning of the processor will be explained in detail with regard to FIG. 3. The output signal 28 may either be evaluated prior to display or may be evaluated once the output signal is displayed at the display 20. In some embodiments the evaluation may be done by passing the output signal 28 through feedback control 30 as illustrated by the arrow 32. The feedback control 30 may evaluate the output signal 28, if the output signal 28 is capable of enhancing the visibility of the ablation to acceptable regions, then the output signal 28 may be displayed at the display 20 as indicated by the arrow 34. Else, the dynamic curve may be altered and applied again to the input image data 24. This process of evaluation may continue till a suitable dynamic curve has been identified for the input image data 24.

Turning now to FIG. 3, the functioning of the processor 26 is explained in detail, in accordance with aspects of the present technique. In the illustrated embodiment, at block 36 the processor 26 detects the level of the acquired input image data 24. As discussed above, the image data 24 may either consist of the entire image, or only a portion of the image about the ablation region. Subsequently, at block 38 a suitable dynamic gain curve is selected for the detected image data. In some embodiments, the dynamic gain curve may be selected from an existing library. In other embodiments, the dynamic gain curves may be manually selected from a database. In alternate embodiments, the dynamic gain curve may be manually selected. At block 40, the dynamic gain curve is applied to the image data. In these embodiments, the dynamic gain curve applied to the image data may be changed depending on the enhancement of the visibility of the ablation region to achieve contrast between the ablated and non-ablated regions.

Turning now to FIGS. 4-6, the illustrated graphs represent exemplary dynamic gain curves that may be applied to the image data. It should be noted that the graphs represented in FIGS. 4-6 are for illustrative purposes only and are not necessarily indicative of an actual curve used for the purposes of enhancing the visibility of the ablation region.

In the illustrated embodiment of FIG. 4, the graph 42 illustrates the transformation of the input image data 44 to the output signal 46 upon application of the dynamic gain curve 48. As illustrated, the dynamic gain curve 48 uses a large range 50 of the output signal 46 to display the low-level image data 44 in the region 52. Assuming that the backscatter intensity from ablation region falls within the range indicated by region 52, the gain curve in FIG. 4 may be employed to enhance the low level signals, regardless of whether or not the signals resulted from ablation. In embodiments where the backscatter intensity from the ablation falls within the region 52, the signals may be displayed over a larger range of the output, thereby substantially increasing the visibility of the ablation region. In certain embodiments, the images may be analyzed to identify where along the input image data 44 the backscatter intensity corresponding to the ablation signal lies. For example, the images may be analyzed by taking the difference between images at different times during the ablation to determine the change in backscatter intensity for a particular region due to the ablation, and then amplifying the display of that region by applying the appropriate gain curve.

In the illustrated embodiment of FIG. 5, the graph 54 illustrates a dynamic gain curve 56 which is configured to display high-level image data in the range 58 by using most of the output signal 46 as illustrated by the arrow 60.

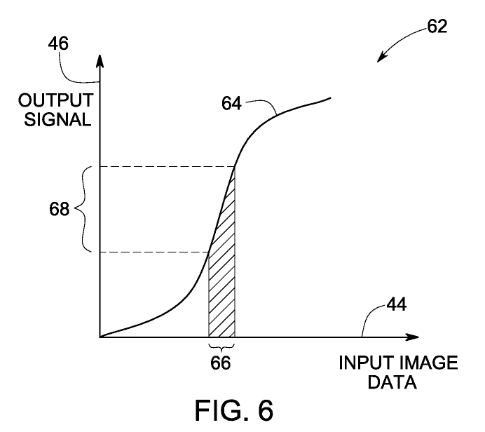

In the illustrated embodiment of FIG. 6, the graph 62 employs a dynamic gain curve 64. To apply the curve 64 to the image data, it is assumed that the backscatter intensity falls substantially within the range 66 of the input image data axis 44. The dynamic gain curve 64 has the steepest slope in the region 66 having the ablation data. The input image data 44 in the range 66 is then transformed into output signal 46 in the region 68, thereby increasing the contrast between the ablated and non-ablated regions.

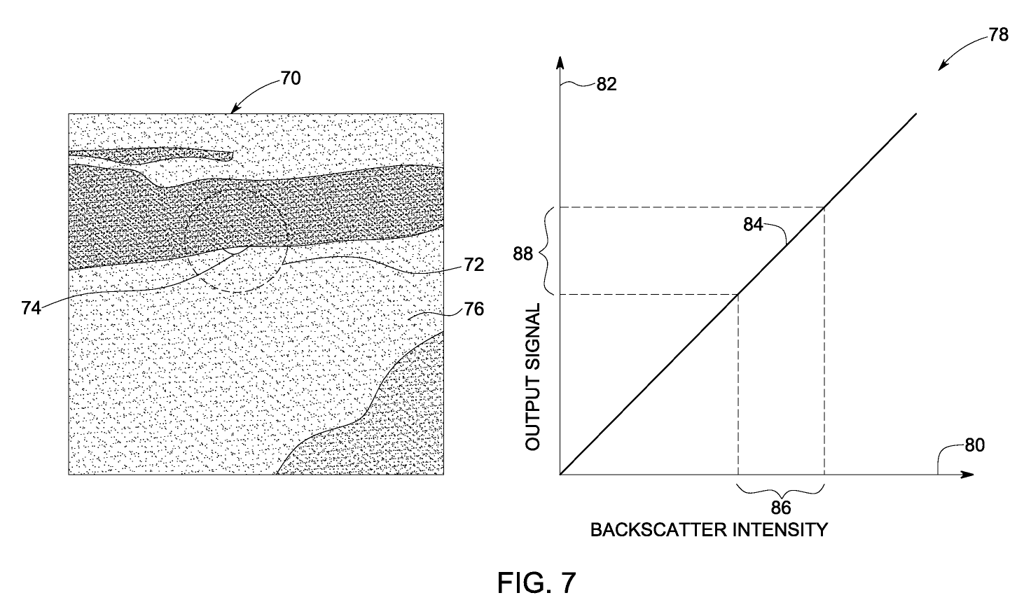

Referring now to FIGS. 7-9 the change in visibility in an ablation region 72 within an anatomical portion 70 of an organ is represented. As will be appreciated, the embodiments of FIGS. 7-9 are for illustrative purposes and various alternatives of the illustrated embodiments are considered within the scope of the present technique. In some embodiments, the image data from one or more image frames may be processed to identify the appropriate input levels corresponding to the ablating tissue. In other embodiments, the ablation region may be tracked by locating the catheter tip. In order to enhance the visibility of the ablated tissue 74 in the region 72 three different dynamic gain curves are applied to the region 72 or to the entire portion 70 to study the change in contrast between the ablation region 74 and the non-ablated region 76. As illustrated in FIGS. 7-9, the different dynamic gain curves affect the backscatter intensity in a different way, thereby affecting the contrast in the ablation region 72 and the non-ablation region 76. In one embodiment, the schematics illustrated in FIGS. 7-9 may correspond to a live image, where the live image is generated based on image data acquired in real-time.

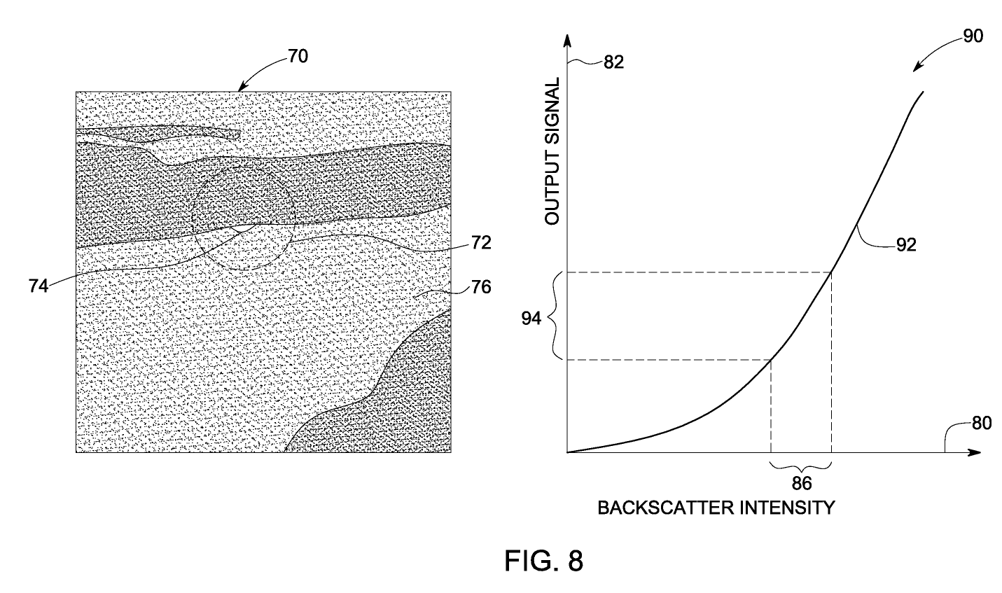

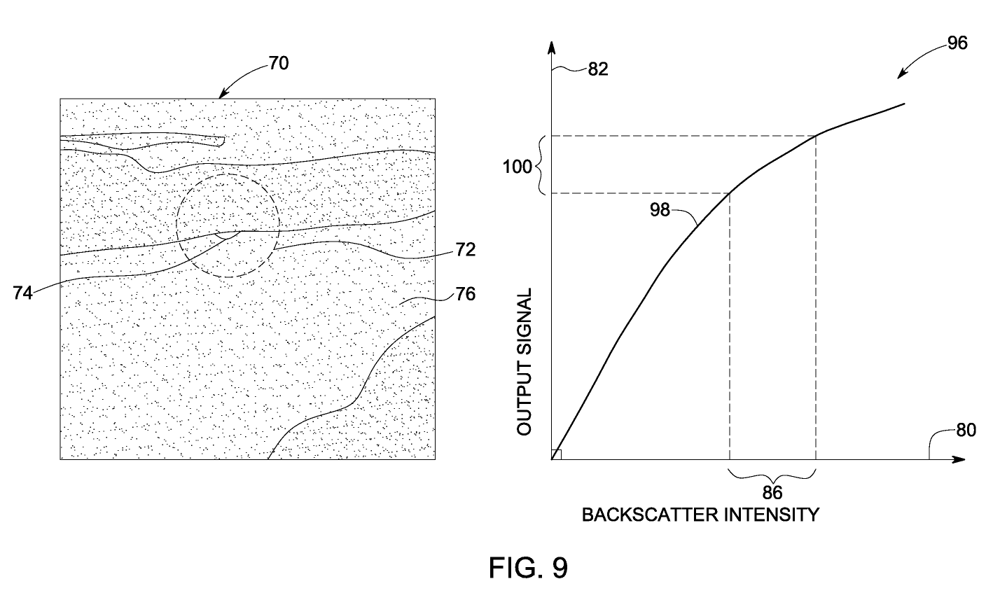

Referring now to the graph 78 in FIG. 7, the x-axis 80 represents the input backscatter intensity, and the y-axis 82 represents the output signal displayed. The dynamic gain curve 84 is applied to the backscatter intensity in the range 86 to generate the output signal as indicated by the reference numeral 88. It should be noted that it is predetermined that the backscatter intensity falls within the range 86. Whereas, as illustrated in the graph 90 (see FIG. 8), while applying the dynamic gain curve 92 for the same range 86 of the backscatter intensity, the output signal falls in the range 94. On application of the curve 92 to the backscatter intensity in the range 86, relatively more of the output signal is dedicated to displaying the input image data as compared to FIG. 7. Accordingly, an increase in the contrast between the ablation 74 and non-ablation regions 76 is observed as illustrated in the schematic of FIG. 8. On the contrary, as illustrated in the graph 96 of FIG. 9, while applying the dynamic gain curve 98 for the backscatter intensity in the range 86, the output signal lies in the range 100. As illustrated, due to the shape of the gain curve 96, the entire image 70 appears bright, thereby reducing the contrast between the ablation 74 and non-ablation regions 76. As will be appreciated, in the illustrated embodiment, relatively less of the output signal is used to display the backscatter intensity corresponding to the ablation signal. As described above, the different dynamic gain curves 84, 92 and 98 could either be manually selected once the image is generated, or the dynamic curves may be selected by the processor based on the feedback circuit.

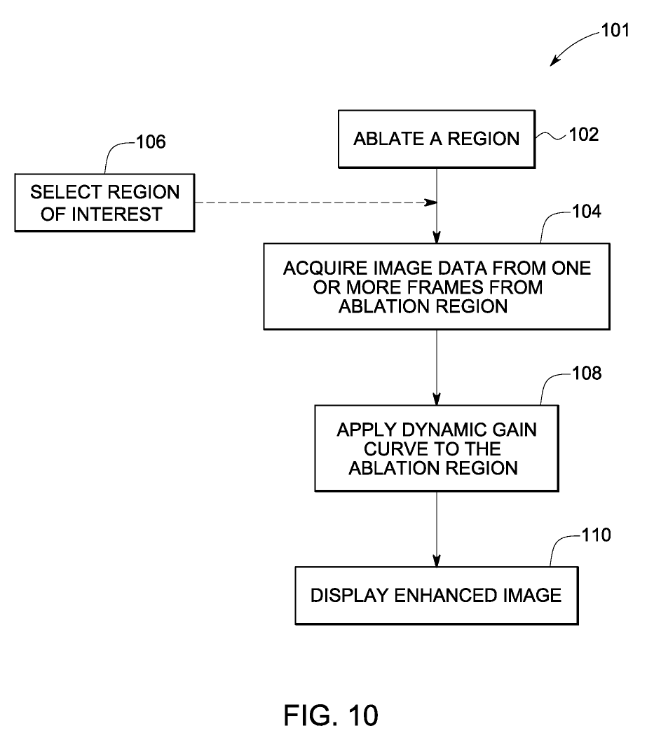

FIG. 10 illustrates an exemplary method 101 for applying a dynamic gain curve to image data acquired from an ablation region. At block 102, a region is being ablated. Subsequently, at block 104, input image data is acquired containing the ablation region in one or more frames. The data from the various frames may then be processed to determine a region from where to select image data to apply the dynamic gain curve. Optionally, at block 106, a region of interest containing the ablation region may be manually selected and the image data from this region of interest may be used to apply the gain curve to enhance the visibility of the ablation region. Alternatively, the image data may be recorded from the entire image. At block 108, once the image data is obtained, the dynamic gain curve may be applied to obtain an output signal. As described above with regard to FIGS. 2 and 3, the dynamic gain curve may either be manually selected or the dynamic gain curve may be selected automatically. Further, a feedback loop may be employed to evaluate the output signal and assess if the enhancement is at permissible level, or if a different dynamic curve is required for the image data. At block 110, the enhanced image is displayed by the system.

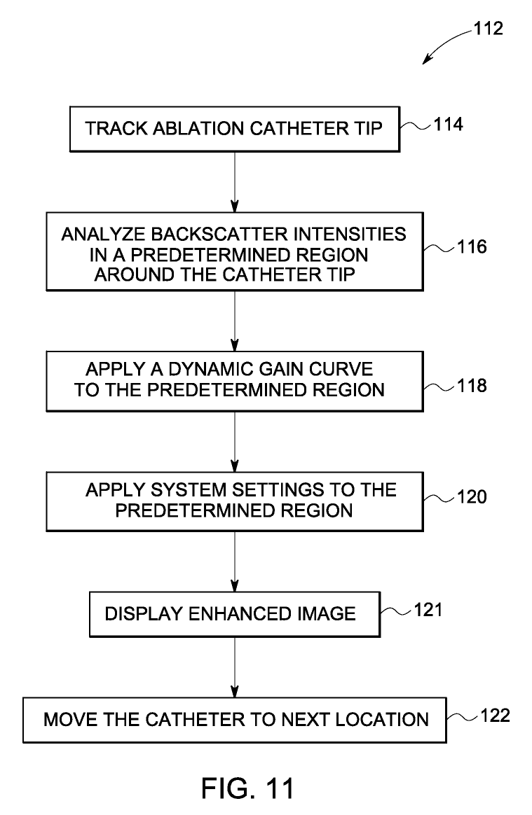

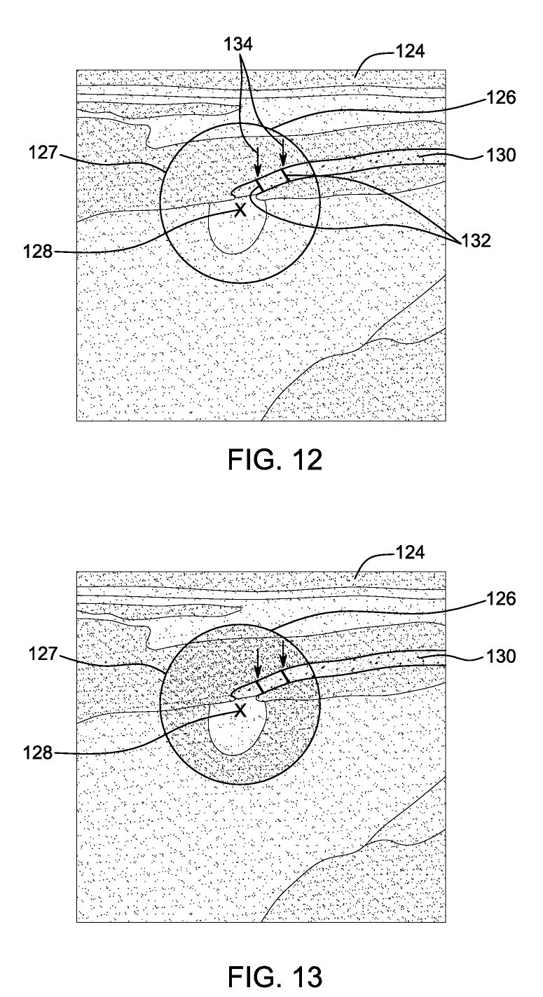

FIG. 11 illustrates a method 112 for enhancing the visibility of an ablation region by tracking a tip of the catheter and thereby locating the ablation region. At block 114, a tip of the catheter is located. Subsequently, a predetermined region around the catheter tip is marked for monitoring the visibility. Alternatively, a region of interest may be manually selected around catheter tip. As will be described in detail with regard to FIGS. 12 and 13, the catheter tip may be located by employing methods, such as but not limited to speckle tracking algorithms associated with the backscatter produced by a catheter, its electrodes, markers or dyes. At block 116, backscatter intensity is analyzed from the region around the catheter tip. At block 118, a dynamic gain curve is applied to the image data from the region around the catheter tip. At block 120, the system settings configured to facilitate application of the dynamic gain curve to the image data are either applied to the selected region. At block 121, the enhanced image is displayed by the system. Once the visibility of the selected region has been enhanced by adjusting the system settings, the catheter tip is moved to the next location for delivering therapy (block 122). The therapy may be delivered by ablating at least a portion of the next location.

FIGS. 12 and 13 illustrate an anatomical portion 124 having an ablation region 126 before and after applying the dynamic gain curve, respectively. The backscatter intensities are recorded from the region 126 marked with a circle 127. The ablated tissue inside the ablation region is depicted by the reference numeral 128. In addition to applying the dynamic gain curve to FIG. 12 to enhance the visibility of the ablation region as shown in FIG. 13, the system settings may also be changed to further enhance the visibility of the ablation region. The ablation catheter is depicted by the reference numeral 130. In the presently contemplated embodiment, the tip of the ablation catheter coincides with the ablated tissue 128 and is depicted by the mark “x”.

The system tracks the location of the catheter tip (“x” mark). The tip of the catheter 130 may be located by various methods. For example, the tracking of the catheter tip may include speckle tracking or other correlation based methods. Electrodes or markers that have a known geometrical relationship (e.g., a known spacing) on the catheter tip may assist in allowing the ultrasound system to track the location of the tip. In one embodiment, the ablation catheter 130 may optionally include a position sensor disposed on the tip of the catheter 130. The position sensor may be configured to track the change in position of the catheter 130 within the anatomy of the patient. Subsequently, the imaging system may be configured to acquire the location information from the position sensor to track the tip of the catheter. In one embodiment, location information may be obtained from the position sensor by localization of the position sensor with respect to fixed points. For example, electromagnetic and/or optical ranging from fixed points, such as fixed sources, reflectors or transponders may be utilized to acquire the location information. Alternatively, in certain other embodiments, location information from the position sensor may be obtained via integration of velocity or acceleration changes from a known reference point. For example, mechanical gyroscopes or optical gyroscopes that respond to changes in velocity and/or acceleration may be employed to obtain the location information from the position sensor.

In the presently contemplated embodiment, the ablation catheter 130 employs markers 132. The markers are indicated by the arrows 134 on the display and the located tip of the catheter 130 is tracked. Once the tip of the catheter 130 has been located, the dynamic gain curve may be applied by the imaging system to ablation region 126. Further, the system settings may be configured to enhance the visibility of the ablation region. Further, automatically selected display settings could be applied to the entire image, or only to the portion in the predetermined region.

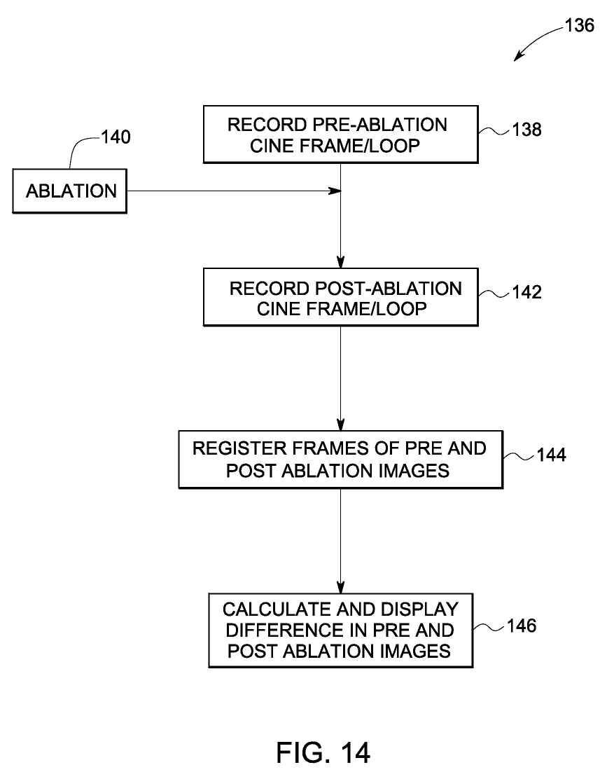

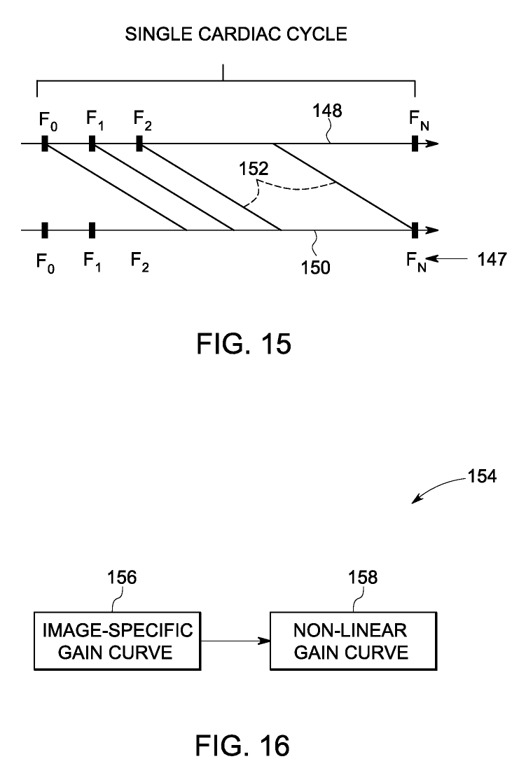

FIG. 14 illustrates a method 136 of enhancing the visibility of an ablation region. At block 138, pre-ablation image frames are recorded for the region that is identified for ablation (at block 138). At block 140, the identified region is ablated. At block 142, post-ablation frames are recorded for the ablated region. At block 144, the frames of the pre-ablation and post-ablation images are registered as will be described in detail with regard to FIG. 15. At block 146, the differences between the backscatter properties of the pre and post ablation images are calculated and displayed to allow better contrast between the ablated and non-ablated region, thereby enhancing the visibility of the ablation region. Further, once the therapy is delivered at one ablation region, the catheter may be moved to another location. As will be appreciated, the movement of the catheter relative to the ablation region may cause difficulty in registering the frames, however, if three dimensional (3D) image data is acquired, registration of pre and post ablation frames may occur between different slices through the 3D volume to enhance the visibility of the ablation region.

As illustrated in the embodiment of FIG. 15, pre and post ablation frames 148 and 150, respectively are recorded. For example, each of the pre and post-ablation frames 148 and 150 may be recorded during two separate single cardiac cycles. Symbols f1, f2, f3, f4, . . . fn represent different frame numbers 147 during registration of the image in a single cardiac cycle. Subsequently, the two images in the frames 148 and 150 are registered. In certain embodiments, registration may include aligning the pre-ablation and post-ablation frames as illustrated by the dotted lines 152. The registration may be done by employing correlation-based methods.

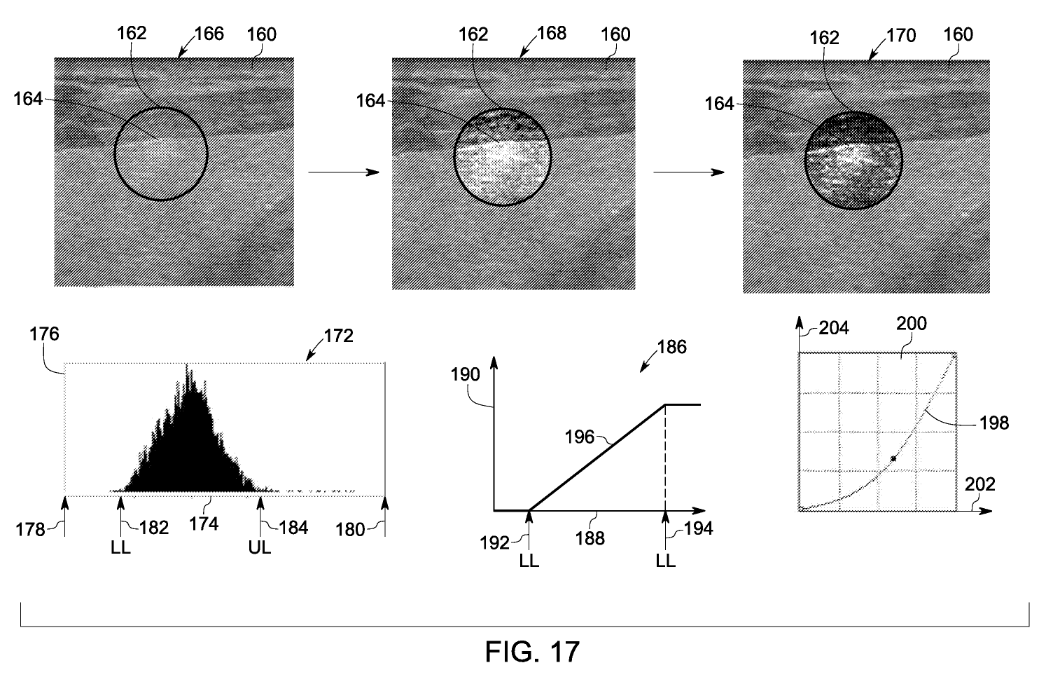

As illustrated in FIG. 16, the gain curve may be divided into two sub-parts for applying to the input image data. In the illustrated embodiment, the gain curve 154 is divided into image-specific gain curve 156 and a non-linear gain curve 158. The image-specific gain curve 156 is applied to selectively expand the input backscatter intensities to fill the entire available output image greylevels or backscatter intensity. The first step of applying the image-specific gain curve 156 uses information from the image, or sub-image representing the region of interest containing the ablation site, to effectively increase the contrast of the lesion. As described with regard to FIG. 17, once the image-specific gain curve 156 has been applied, the second step includes applying one or more non-linear gain curves 158 designed to further increase the contrast of the lesion to all the frames. The non-linear gain curve 158 is applied after the image-specific gain curve 156, so it is assumed the region of interest spans the full set of output greylevels or backscatter intensity in which the non-linear gain curve 158 is applied. A number of different curves may be used, such as the non-linear curves shown in FIGS. 4-6. These non-linear curves may increase the contrast for the darkest (see FIG. 4), brightest (see FIG. 5), or midrange (see FIG. 6) backscatter intensity. The amount of increase in the contrast for these regions is controlled by the slope of the curves. The slope of the curves may be adjusted as a user-selectable parameter on the ultrasound system.

As illustrated in FIG. 17, the change in visibility in an ablation region 162 having an ablated tissue 164 within an anatomical portion 160 of an organ is represented. In the illustrated embodiment, image data from the ablation region 162 is used to create a histogram 172. As illustrated, the backscatter intensity is represented on the x-axis 174 and the number of counts at each intensity is represented on the y axis 176. The original image data falls within the arrows 178 and 180. The input image data that is used to represent the entire range of output greylevels is then confined within the arrows 182 and 184. The arrows 182 and 184 represent the lower limit of 5^{th} and the upper limit of 95^{th} percentiles for the histogram, respectively. The specified percentiles in the histogram chosen to correspond to the full output greylevels range may be varied depending on the images to reduce sensitivity to a small number of outlier pixels with very low or very high values for the backscatter intensity. This information may then be used to generate image-specific gain curve 196 of graph 186. The image-specific gain curve 196 is applied to the image 166 to expand the contrast and obtain image 168. The image-specific gain curve 196 includes x-axis 188 representing the backscatter intensity of the input image data, and the y-axis 190 representing the output image data. The lower limit (LL) 192 and the upper limit (UL) 194 obtained from the histogram 172 are then applied to obtain the image 168. Subsequently, a non-linear gain curve, such as the curve 198 of the graph 200 is applied to obtain the image 170 that has a higher contrast in the region of interest 162 as compared to image 168. In the graph 200, the x-axis 202 represents the input backscatter intensity, and the y-axis 204 represents the output signal displayed. The non-linear gain curve 198 is applied to further enhance the contrast of the region 162. As will be appreciated, the percentiles in the histogram 172 and the graph 200 are exemplary embodiments and may be varied depending on the specific images.

As will be appreciated by those of ordinary skill in the art, the foregoing example, demonstrations, and process steps may be implemented by suitable code on a processor-based system, such as a general-purpose or special-purpose computer. It should also be noted that different implementations of the present technique may perform some or all of the steps described herein in different orders or substantially concurrently, that is, in parallel. Furthermore, the functions may be implemented in a variety of programming languages, including but not limited to C++ or Java. Such code, as will be appreciated by those of ordinary skill in the art, may be stored or adapted for storage on one or more tangible, machine readable media, such as on memory chips, local or remote hard disks, optical disks (that is, CD's or DVD's), or other media, which may be accessed by a processor-based system to execute the stored code. Note that the tangible media may comprise paper or another suitable medium upon which the instructions are printed. For instance, the instructions can be electronically captured via optical scanning of the paper or other medium, then compiled, interpreted or otherwise processed in a suitable manner if necessary, and then stored in a computer memory.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method for imaging during ablation procedures using ultrasound imaging, comprising:
obtaining input image data about an ablation region, wherein the image data comprises backscatter intensity; and
applying a dynamic gain curve based on the image data to obtain an output signal for use in enhancing the visibility of the ablation region.

2. The method of claim 1, further comprising processing the image data from one or more image frames to identify changes in localized backscatter properties due to ablation in the ablation region.

3. The method of claim 2, wherein the processing comprises integrating the image data from the one or more image frames to account for the spatial movement of ablated tissues.

4. The method of claim 2, wherein the processing comprises calculating regional differences in the input backscatter intensity of the one or more frames.

5. The method of claim 1, wherein the dynamic gain curve is a relationship between the input backscatter intensity and a displayed output signal.

6. The method of claim 1, wherein the ablation region is ablated by employing one or more of ethanol, liquid nitrogen, ultrasound, radiofrequency and cryogenic ablation.

7. The method of claim 1, wherein the dynamic gain curve is applied in a region of a displayed image to enhance visibility corresponding to the region.

8. The method of claim 1, wherein the dynamic gain curve is applied in a region of interest comprising an ablated tissue.

9. The method of claim 1, further comprising selecting a region of interest within the region by employing a user interface.

10. The method of claim 1, further comprising tracking a tip of a catheter to locate the ablation region.

11. The method of claim 10, wherein tracking the tip of the catheter comprises employing correlation based methods.

12. The method of claim 10, wherein tracking the tip of the catheter comprises employing an electrode having predetermined geometrical relationship with the catheter tip.

13. The method of claim 1, wherein obtaining image data comprises:
acquiring pre-ablation and post-ablation images; and
registering the pre-ablation and post-ablation images frame by frame.

14. The method of claim 13, further comprising calculating and displaying differences in pre and post ablation images.

15. The method of claim 1, wherein the ultrasound imaging includes one or more of an intracardiac probe, a transesophageal probe, a transthoracic probe, or combinations thereof.

16. The method of claim 1, wherein the ultrasound imaging includes internal ablation.

17. A method for enhancing the visibility of an ablation region during ablation procedures, comprising:
processing backscatter data from one or more image frames to identify changes in localized regions of image data; and
applying a dynamic gain curve to obtain an enhanced output signal from the ablation region.

18. The method of claim 17, wherein the dynamic gain curve is applied to a region of interest comprising an ablated tissue.

19. The method of claim 17, further comprising adjusting the system settings to enhance the visibility of the ablation region.

20. The method of claim 19, wherein the system settings are applied to an area of a displayed image, or a region of interest located within the area of a displayed image.

21. The method of claim 20, wherein a user interface device is employed to define the region of interest.

22. The method of claim 17, wherein processing comprises generating processed backscatter data.

23. A method for in-situ enhancement of the visibility of an ablation region, comprising:
monitoring the ablation region;
tracking a location of a catheter tip during ablation in the ablation region;
analyzing a backscatter intensity in a predetermined region around the catheter tip; and
adjusting the system settings to obtain enhanced backscatter data from the predetermined region.

24. The method of claim 23, wherein tracking the location comprises employing correlation methods, geometrical shapes relation, electrodes, markers, or combinations thereof.
